(19) United States
(12) Patent Application Publication
(10) Pub. No.: US 2023/0056782 A1
(43) Pub. Date: Feb. 23, 2023

(54) ANTIBODIES, PHARMACEUTICAL COMPOSITIONS AND USES THEREOF

(71) Applicants: Ming-Tain LAI, Taipei (TW); Jiann-Shiun LAI, Taipei (TW); Chiu-Chun LIN, Taipei (TW); I-Ju CHEN, Taipei (TW); Woan-Eng CHAN, Taipei (TW); OBI PHARMA, INC., Taipei (TW)

(72) Inventors: MING-TAIN LAI, Taipei (TW); JIANN-SHIUN LAI, Taipei (TW); CHIU-CHUN LIN, Taipei (TW); I-JU CHEN, Taipei (TW); WOAN-ENG CHAN, Taipei (TW)

(21) Appl. No.: 17/789,796

(22) PCT Filed: Dec. 30, 2020
(86) PCT No.: PCT/US20/67391

§ 371 Date: Jun. 29, 2022

Related U.S. Application Data
(60) Provisional application No. 62/954,669, filed on Dec. 30, 2019.

(51) Int. Cl. C07K 16/18; A61P 35/00

(52) U.S. Cl. CPC C07K 16/18; A61P 35/00; C07K 2317/622; C07K 2317/55; C07K 2317/24; C07K 2317/31

(57) ABSTRACT

The present invention provides antibodies or the antigen-binding portion thereof, that bind to stage-specific embryonic antigen 3 (SSEA-3) antigen. Also disclosed herein are pharmaceutical compositions and methods for the inhibition of cancer cells in a subject in need thereof. The pharmaceutical compositions include an antibody or an antigen-binding portion thereof and at least one pharmaceutically acceptable carrier.

### CROSS-REFERENCE TO RELATED APPLICATIONS

This application claims priority of Provisional Application No. 62/954,669, filed on Dec. 30, 2019, the content of which is incorporated herein in its entirety by reference.

### FIELD

The present invention relates to antibodies to tumor-associated carbohydrate antigens, including specific portions or variants specific for at least one tumor-associated carbohydrate antigen or fragment thereof, as well as nucleic acids encoding such antibodies, complementary nucleic acids, vectors, host cells and methods of making and using thereof, including therapeutic formulations and pharmaceutical compositions comprising the antibody. Further, methods are provided for administering antibodies to a subject in an amount effective to inhibit cancer cells.

### SEQUENCE LISTING

The instant application contains a Sequence Listing which has been submitted electronically in ASCII format and is hereby incorporated by reference in its entirety. Said ASCII copy, created Dec. 1, 2020, is named “OBIP-1PCT_SEQList_ST25.txt” and is 4,740 bytes in size.

### BACKGROUND

Numerous surface carbohydrates are expressed in malignant tumor cells. For example, Globo H (Fuc α1->2Galβ1->3GalNAcβ1->3Gal α1->4Galβ1->4Glc) has been shown to be overexpressed on a variety of epithelial cancers and is associated with tumor aggressiveness and poor prognosis in breast cancer and small cell lung carcinoma. Previous studies have shown that Globo H and stage-specific embryonic antigen 3 (Galβ1→3GalNAcβ1→3Galα1→4Galβ1→4Glcβ1) (SSEA-3, also called Gb5) were observed on breast cancer cells and breast cancer stem cells (Chang W W et al., (2008) PNAS, 105(33):11667-11672; Cheung S K et al., (2016) PNAS, 113(4):960-965). In addition, SSEA-4 (stage-specific embryonic antigen-4) (Neu5Acα2→3Gal β1→3GalNAcβ1→3Galα1→4Galβ1→4Glcβ1) has been commonly used as a cell surface marker for pluripotent human embryonic stem cells and has been used to isolate mesenchymal stem cells and enrich neural progenitor cells (Kannagi R et al., (1983) EMBO J, 2:2355-2361). These findings support that Globo series antigens (Globo H, SSEA-3, and SSEA-4) are unique targets for cancer therapies and can be used to direct therapeutic agents in targeting cancer cells effectively.

These findings support a rationale for the development of antibodies to tumor associated carbohydrate antigens, as there is still an unmet need for effective treatment and/or prevention for cancer. The present invention provides antibodies to tumor associated carbohydrate antigens to satisfy these and other needs.

### SUMMARY OF THE INVENTION

The present invention provides for antibodies, or antigen-binding portions thereof, comprising a variable domain that bind to a carbohydrate antigen, conjugated versions of these antibodies, encoding or complementary nucleic acids, vectors, host cells, compositions, formulations, devices, transgenic animals, transgenic plants related thereto, and methods of making and using thereof, as described and enabled herein, in combination with what is known in the art. The antibody or antigen-binding portion thereof may have a dissociation constant (K_{D}) of about 10E-7 M or less, about 10E-8 M or less, about 10E-9 M or less, about 10E-10 M or less, about 10E-11 M or less, or about 10E-12 M or less. The antibody or antigen-binding portion thereof may be humanized or chimeric.

In yet another embodiment, the present invention provides for an antibody, or an antigen-binding portion thereof, including a heavy chain variable domain having an amino acid sequence with about 80% to about 100% identity to the amino acid sequence shown in SEQ ID NO: 3; and a light chain variable domain having an amino acid sequence with about 80% to about 100% identity to the amino acid sequence shown in SEQ ID NO: 4.

In some embodiments, an antibody, or an antigen-binding portion thereof, includes a heavy chain region, wherein the heavy chain region includes a CDR having an amino acid sequence with about 80% to about 100% identity to the amino acid sequence selected from the group consisting of SEQ ID NOs: 5, 6, and 7. In other embodiments, an antibody, or an antigen-binding portion thereof, includes a light chain region, wherein the light chain region includes a CDR having an amino acid sequence with about 80% to about 100% identity to the amino acid sequence selected from the groups consisting of SEQ ID NOs: 8, 9, and 10.

The present invention provides a pharmaceutical composition including the antibody or antigen-binding portion thereof as described herein and at least one pharmaceutically acceptable carrier.

The present invention also provides for a method of inhibiting Globo H expressing cancer cells, comprising administering to a subject in need thereof an effective amount of the antibody or antigen-binding portion thereof described herein, wherein the Globo H expressing cancer cells are inhibited.

The present invention also provides for hybridoma clones designated as 1E12b (deposited under American Type Culture Collection (ATCC) Accession Number PTA-126149, and antibodies or antigen-binding portions produced therefrom.

### BRIEF DESCRIPTION OF THE DRAWINGS

FIG. 1 shows the characterization of the binding affinity of mouse anti-SSEA3 antibody (IgM-1E12b).

FIG. 2 shows flow cytometry histograms. All the testing cells were stained with a commercial anti-SSEA3 antibody (MC-631, BioLegend, Cat No. 330302, red line), mouse anti-SSEA3 antibody (IgM-1E12b, green line) and isotype control (black line) followed by incubation with PE or FITC-conjugated secondary antibody. (FIG. 2A) MCF-7 cells (FIG. 2B) SKOV cells and (FIG. 2C) SKBR3 cells.

FIG. 3 shows a cross reactivity test with biotinylated sugars by chemiluminescent sandwich ELISA analysis.

### DETAILED DESCRIPTION OF THE INVENTION

As used herein, the articles “a” and “an” refer to one or more than one (i.e., at least one) of the grammatical object of the article. By way of example, “an element” means one element or more than one element.

An “effective amount,” as used herein, refers to a dose of the vaccine or pharmaceutical composition that is sufficient to reduce the symptoms and signs of cancer, such as weight loss, pain and palpable mass, which is detectable, either clinically as a palpable mass or radiologically through various imaging means. The term “effective amount” and “therapeutically effective amount” are used interchangeably.

The term “subject” can refer to a vertebrate having cancer or to a vertebrate deemed to be in need of cancer treatment. Subjects include all warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

A “combination” refers to combination therapy would be the amount of the antibody and/or the amount of other chemotherapeutic agent, photodynamic therapeutic agent or biological agent that when administered together (either as co-administration and/or co-formulation), either sequentially or simultaneously, on the same or different days during a treatment cycle, have a synergistic effect that is therapeutically effective and more than therapeutically additive.

All numbers herein are approximations and may be modified by “about.”

The present invention provides for pharmaceutical compositions and methods for the treatment or inhibition of cancer cells. The pharmaceutical compositions include antibodies that recognize carbohydrate antigen, including mouse monoclonal antibodies, humanized antibodies, chimeric antibodies, or antigen-binding portions of any of the foregoing. These antibodies (or antigen-binding portion thereof) can neutralize the carbohydrate antigen, and/or inhibit cancer cells. Therefore, the present antibodies or antigen-binding portion thereof can be used in the treatment or inhibition of cancer cells.

Antibodies of the present invention include any protein or peptide that comprise at least one complementarity determining region (CDR) of a heavy or light chain, or a ligand binding portion thereof, derived from an antibody produced by the hybridoma designated 1E12b (deposited on Nov. 19, 2019 at the American Type Culture Collection (“ATCC”), 10801 University Boulevard, Manassas, Va. 20110-2209 and having ATCC Accession No.: PTA-126149) as described herein. (All ATCC deposits recited herein were made under the Budapest Treaty). Antibodies include antibody fragments, antibody variants, monoclonal antibodies, polyclonal antibodies, and recombinant antibodies and the like. Antibodies can be generated in mice, rabbits, or humans.

Antibodies of the present invention also include chimerized or humanized monoclonal antibodies generated from antibodies of the present invention.

Thus, anti-cancer antibodies of the present invention include in combination with a heavy chain or light chain variable region, a heavy chain or light chain constant region, a framework region, or any portion thereof, of non-murine origin, preferably of human origin, which can be incorporated into an antibody of the present invention.

Antibodies of the present invention are capable of modulating, decreasing, antagonizing, mitigating, alleviating, blocking, inhibiting, abrogating and/or interfering with at least one SSEA-3 expressing cancer cell activity in vitro, in situ and/or in vivo.

The term “antibody” is further intended to encompass antibodies, digestion fragments, specified portions and variants thereof, including antibody mimetics or comprising portions of antibodies that mimic the structure and/or function of an anti-cancer antibody or specified fragment or portion thereof, including single-chain antibodies and fragments thereof, each containing at least one CDR derived from an anti-cancer antibody of the present invention. Functional fragments include antigen-binding fragments that bind to an SSEA-3 expressing cancer cells. For example, antibody fragments capable of binding to SSEA-3 expression cancer cells or portions thereof, including, but not limited to Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

As used herein, 1E12b refers to the hybridoma clone or the antibodies generated by the corresponding hybridoma clone.

An antigen-binding portion of an antibody may include a portion of an antibody that specifically binds to a carbohydrate antigen (e.g., Globo H, SSEA-3 or SSEA-4).

The humanized antibody of the present invention is an antibody from a non-human species where the amino acid sequence in the non-antigen binding regions (and/or the antigen-binding regions) has been altered so that the antibody more closely resembles a human antibody while retaining its original binding ability.

Humanized antibodies can be generated by replacing sequences of the variable region that are not directly involved in antigen binding with equivalent sequences from human variable regions. Those methods include isolating, manipulating, and expressing the nucleic acid sequences that encode all or part of variable regions from at least one of a heavy or light chain. Sources of such nucleic acid are well known to those skilled in the art. The recombinant DNA encoding the humanized antibody, or fragment thereof, can then be cloned into an appropriate expression vector.

An antibody light or heavy chain variable region comprises a framework region interrupted by three hypervariable regions, referred to as CDRs. In one embodiment, humanized antibodies are antibody molecules from non-human species having one, two or all CDRs from the non-human species and one, two or all three framework regions from a human immunoglobulin molecule.

According to one aspect of the invention, the location of the CDRs and framework residues are determined by methods disclosed in Kabat, E. A. et al., (1991) Sequences of Proteins of Immunological Interest, Fifth Edition, U.S. Department of Health and Human Services, NIH Publication No. 91-3242. According to another aspect of the invention, the antibody or the antigen-binding portion thereof may have the following structure:
Leader Sequence—FW1-CDR1-FW2-CDR2-FW3-CDR3-
   in which the framework regions FW1, FW2, FW3, and CDRs CDR1, CDR2, CDR3 have amino acid sequences disclosed in Table 1.

The humanized antibodies of the present invention can be produced by methods well known in the art. For example, once non-human (e.g., murine) antibodies are obtained, variable regions can be sequenced, and the location of the CDRs and framework residues determined. Kabat, E. A. et al., (1991) Sequences of Proteins of Immunological Interest, Fifth Edition, U.S. Department of Health and Human Services, NIH Publication No. 91-3242. Chothia, C. et al., (1987) J. Mol. Biol., 196:901-917. The DNA encoding the light and heavy chain variable regions can, optionally, be ligated to corresponding constant regions and then subcloned into an appropriate expression vector. CDR-grafted antibody molecules can be produced by CDR-grafting or CDR substitution. One, two, or all CDRs of an immunoglobulin chain can be replaced. For example, all of the CDRs of a particular antibody may be from at least a portion of a non-human animal (e.g., mouse such as CDRs shown in Table 1) or only some of the CDRs may be replaced. It is only necessary to keep the CDRs required for binding of the antibody to a predetermined carbohydrate antigen (e.g., Globo H) (Morrison, S. L., (1985) Science, 229:1202-1207; Oi et al., (1986) BioTechniques, 4:214; U.S. Pat. Nos. 5,585,089; 5,225,539; 5,693,761 and 5,693,762; EP Patent No 519596; Jones et al., (1986) Nature, 321:552-525; Verhoeyan et al., (1988) Science, 239:1534; Beidler et al., (1988) J. Immunol., 141:4053-4060).

Also encompassed by the present invention are antibodies or antigen-binding portions thereof comprising one or two variable regions as disclosed herein, with the other regions replaced by sequences from at least one different species including, but not limited to, human, rabbits, sheep, dogs, cats, cows, horses, goats, pigs, monkeys, apes, gorillas, chimpanzees, ducks, geese, chickens, amphibians, reptiles and other animals.

A chimeric antibody is a molecule in which different portions are derived from different animal species. For example, an antibody may contain a variable region derived from a murine mAb and a human immunoglobulin constant region. Chimeric antibodies can be produced by recombinant DNA techniques (Morrison, et al., (1984) PNAS, 81:6851-6855). For example, a gene encoding a murine (or other species) antibody molecule is digested with restriction enzymes to remove the region encoding the murine Fc, and the equivalent portion of a gene encoding a human Fc constant region is then substituted into the recombinant DNA molecule. Chimeric antibodies can also be created by recombinant DNA techniques where DNA encoding murine V regions can be ligated to DNA encoding the human constant regions (Better et al., (1988) Science, 240:1041-1043; Liu et al., (1987) PNAS, 84:3439-3443; Liu et al., (1987) J. Immunol., 139:3521-3526; Sun et al. (1987) PNAS, 84:214-218; Nishimura et al., (1987) Canc. Res., 47:999-1005; Wood et al., (1985) Nature, 314:446-449; Shaw et al., (1988) J. Natl. Cancer Inst., 80:1553-1559); International Patent Publication Nos. WO1987002671 and WO 86/01533; European Patent Application Nos. 184,187; 171,496; 125,023; and 173,494; U.S. Pat. No. 4,816,567).

The antibodies can be full-length or can comprise a fragment (or fragments) of the antibody having an antigen-binding portion, including, but not limited to, Fab, F(ab′)2, Fab′, F(ab)′, Fv, single-chain Fv (scFv), bivalent scFv (bi-scFv), trivalent scFv (tri-scFv), Fd, dAb fragment (Ward et al., (1989) Nature, 341:544-546), an isolated CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention (Bird et al., (1988) Science, 242:423-426; Huston et al., (1988) PNAS, 85:5879-5883).

The antibodies or antigen-binding portions thereof of the present invention may be monospecific, bi-specific or multispecific. Multispecific or bi-specific antibodies or fragments thereof may be specific for different epitopes of one target carbohydrate (e.g., Globo H) or may contain antigen-binding domains specific for more than one target carbohydrate (e.g., antigen-binding domains specific for Globo H, SSEA-3, and SSEA-4). In one embodiment, a multispecific antibody or antigen-binding portion thereof comprises at least two different variable domains, wherein each variable domain is capable of specifically binding to a separate carbohydrate antigen or to a different epitope on the same carbohydrate antigen (Tutt et al., (1991) J. Immunol. 147:60-69; Kufer et al., (2004) Trends Biotechnol. 22:238-244). The present antibodies can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity.

All antibody isotypes are encompassed by the present invention, including IgG (e.g., IgG1, IgG2, IgG3, IgG4), IgM, IgA (IgA1, IgA2), IgD or IgE (all classes and subclasses are encompassed by the present invention). The antibodies or antigen-binding portions thereof may be mammalian (e.g., mouse, human) antibodies or antigen-binding portions thereof. The light chains of the antibody may be of kappa or lambda type.

The variable regions of the present antibodies or antigen-binding portions thereof can be from a non-human or human source. The framework of the present antibodies or antigen-binding portions thereof can be human, humanized, non-human (e.g., a murine framework modified to decrease antigenicity in humans), or a synthetic framework (e.g., a consensus sequence).

In one embodiment, the present antibodies, or antigen-binding portions thereof, include at least one heavy chain variable region and/or at least one light chain variable region.

The present antibodies or antigen-binding portions thereof specifically bind to SSEA-3 with a dissociation constant (K_{D}) of less than about 10E-7 M, less than about 10E-8 M, less than about 10E-9 M, less than about 10E-10 M, less than about 10E-11 M, or less than about 10E-12 M. In one embodiment, the antibody or the antibody binding portion thereof has a dissociation constant (K_{D}) of 1˜10×10E-9 or less. In another embodiment, the Kd is determined by surface plasmon resonance.

Antibodies with a variable heavy chain region and a variable light chain region that are at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% identity to the variable heavy chain region and variable light chain region of the antibody produced by clone 1E12b, and can also bind to a carbohydrate antigen (e.g. SSEA-3). Identity can be present at either the amino acid or nucleotide sequence level.

In some embodiments, the antibodies or antigen-binding portions thereof include, for example, the variable heavy chains and/or variable light chains of the antibodies produced by hybridoma 1E12b, are shown in Table 1.

In related embodiments, the antibodies or antigen-binding portions thereof include, for example, the CDRs of the variable heavy chains and/or the CDRs of the variable light chains of the antibodies produced from hybridoma 1E12b. The CDRs of the variable heavy chains and the variable light chains from these hybridoma clones are shown in Table 1.

TABLE 1
SEQ ID NO. 1-10

| Hybridoma | Chain | | SEQ |
|---|---|---|---|
| Clones | Region | Sequence | ID No. |
| 1E12b | Heavy | Nucleic acid Sequence | 1 |
| | Chain | GAGGTGAAGCTTCTCGAGTCTGGA | |
| | Variable | GGTGGCCTGGTGCAGCCTGGAGGA | |
| | Region | TCCCTGAAACTCTCCTGTGCAGCC | |
| | (VH) | TCAGGATTCGATTTTAGTATATAC | |
| | | TGGATGAGTTGGGTCCGGCAGGCT | |
| | | CCAGGGAAAGGGCTAGAATGGATT | |
| | | GGAGAAATTAATCAACATAGCAGT | |
| | | ACGATAAACTATACGCCATCTCTA | |
| | | AAGGATAAGTTCATCATCTCCAGA | |
| | | GACAACGCCAAAAATACGCTGTAC | |
| | | CTGCAAATGAGCAAAGTGAGATCT | |
| | | GAGGACACAGCCCTTTATTACTGT | |
| | | GCAAGACGGTATGGTAACTACGCA | |
| | | GACTACTTTGACTACTGGGGCCAA | |
| | | GGCACCACTCTCACAGTCTCCTCA | |
| 1E12b | Light | Nucleic acid Sequence | 2 |
| | Chain | GAAAATGTGCTCACCCAGTCTCCA | |
| | Variable | GCAATCATGTCTGCATCTCCAGGG | |
| | Region | GAAAAGGTCACCATGACCTGCAGG | |
| | (VL) | GCCAGCTCAAGTGTAAGTTCCAAT | |
| | | TACTTGCACTGGTACCAGCAGAAG | |
| | | TCAGGTGCCTCCCCCAAACTCTGG | |
| | | ATTTATAGCACATCCAACTTGGCT | |
| | | TCTGGAGTCCCTGCTCGCTTCAGT | |
| | | GGCAGTGGGTCTGGGACCTCTTAC | |
| | | TCTCTCACAATCAGCAGTGTGGAG | |
| | | GCTGAAGATGCTGCCACTTATTAC | |
| | | TGCCACCAGTGCAGTACTTACCCA | |
| | | CTCACGTTCGGTGCTGGGACCAAG | |
| | | CTGGAACTGAAACGG | |
| 1E12b | Heavy | Amino Acid Sequence | 3 |
| | Chain | EVKLLESGGGLVQPGGSLKLSCAA | |
| | Variable | SGFDFSIYWMSWVRQAPGKGLEWI | |
| | Region | GEINQHSSTINYTPSLKDKFIISR | |
| | (VH) | DNAKNTLYLQMSKVRSEDTALYYC | |
| | | ARRYGNYADYFDYWGQGTTLTVSS | |
| 1E12b | Light | Amino Acid Sequence | 4 |
| | Chain | ENVLTQSPAIMSASPGEKVTMTCR | |
| | (VL) | ASSSVSSNYLHWYQQKSGASPKLW | |
| | | IYSTSNLASGVPARFSGSGSGTSY | |
| | | SLTISSVEAEDAATYYCHQCSTYP | |
| | | LTFGAGTKLELKR | |
| 1E12b | Heavy | Amino Acid Sequence | 5 |
| | Chain | GFDFSIYWMS | |
| | CDR1 | | |
| 1E12b | Heavy | Amino Acid Sequence | 6 |
| | Chain | EINQHSSTINYTPS | |
| | CDR2 | | |
| 1E12b | Heavy | Amino Acid Sequence | 7 |
| | Chain | RYGNYADYFDY | |
| | CDR3 | | |
| 1E12b | Light | Amino Acid Sequence | 8 |
| | Chain | RASSSVSSNYLHW | |
| | CDR1 | | |
| 1E12b | Light | Amino Acid Sequence | 9 |
| | Chain | STSNLAS | |
| | CDR2 | | |
| 1E12b | Light | Amino Acid Sequence | 10 |
| | Chain | CHQCSTYPL | |
| | CDR3 | | |

The present invention also encompasses a nucleic acid encoding the present antibody or antigen-binding portion thereof that specifically binds to a carbohydrate antigen. In one embodiment, the carbohydrate antigen is SSEA-3. In another embodiment, the carbohydrate antigen is SSEA-4. In yet another embodiment, the carbohydrate antigen is Globo H. The nucleic acid may be expressed in a cell to produce the present antibody or antigen-binding portion thereof.

In certain embodiments, the antibodies or antigen-binding portions thereof include a variable heavy chain region having an amino acid sequence that is with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% identity to SEQ ID NO: 3 (Hybridoma 1E12b).

In certain embodiments, the antibodies or antigen-binding portions thereof include a variable light chain region having an amino acid sequence that is with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% identity to SEQ ID NO: 4 (Hybridoma 1E12b).

In certain embodiments, a variable heavy chain region of the antibodies or antigen-binding portions thereof having an amino acid sequence that is with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% identity to SEQ ID NO: 3, and a variable light chain region of the antibodies or antigen-binding portions thereof includes an amino acid sequence that is with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% identity to SEQ ID NO: 4.

A variable heavy chain region of the antibodies or antigen-binding portions thereof can comprise one, two three or more CDRs include amino acid sequences that are with at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identity to CDRs of the variable heavy chain region of an antibody produced by hybridoma 1E12b (SEQ ID NOs: 5, 6 and 7).

A variable light chain region of the antibodies or antigen-binding portions thereof can comprise one, two three or more CDRs include amino acid sequences that are with at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identity to CDRs of the variable light chain region of an antibody produced by hybridoma 1E12b (SEQ ID NOs: 8, 9 and 10).

A variable heavy chain region of the antibodies or antigen-binding portions thereof can comprise one, two three or more CDRs include amino acid sequences that are with at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identity to CDRs of the variable heavy chain region of an antibody produced by hybridoma 1E12b (SEQ ID NOs: 5, 6, 7), and a variable light chain region of the antibodies or antigen-binding portions thereof can comprise one, two three or more CDRs include amino acid sequences that are with at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identity to CDRs of the variable light chain region of an antibody produced by hybridoma 1E12b (SEQ ID NOs: 8, 9 and 10).

In certain embodiments, the variable regions corresponding to the variable regions in Table 1 have sequence variations. For example, a heavy chain variable region, in which 1, 2, 3, 4, 5, 6, 7 or 8 residues, or less than 40%, less than about 30%, about 25%, about 20%, about 15%, about 10%, about 9%, about 8%, about 7%, about 6%, about 5%, about 4%, about 3%, about 2% or about 1% amino acid residues substituted or deleted but retain essentially the same immunological properties including, but not limited to, binding to a carbohydrate antigen.

In certain embodiments, CDRs corresponding to the CDRs in Table 1 have sequence variations. For example, CDRs, in which 1, 2, 3, 4, 5, 6, 7 or 8 residues, or less than 20%, less than 30%, or less than about 40% of total residues in the CDR, are substituted or deleted can be present in an antibody (or antigen-binding portion thereof) that binds a carbohydrate antigen.

The antibodies or antigen-binding portions may be peptides. Such peptides can include variants, analogs, orthologs, homologs, and derivatives of peptides that exhibit biological activity, e.g., binding of a carbohydrate antigen. The peptides may contain one or more analogs of an amino acid (including, for example, non-naturally occurring amino acids, amino acids which only occur naturally in an unrelated biological system, modified amino acids from mammalian systems, etc.), peptides with substituted linkages, as well as other modifications known in the art.

Also within the scope of the invention are antibodies or antigen-binding portions thereof in which specific amino acids have been substituted, deleted or added. In an exemplary embodiment, these alternations do not have a substantial effect on the peptide's biological properties, such as binding affinity. In another exemplary embodiment, antibodies may have amino acid substitutions in the framework region, such as to improve the binding affinity of the antibody to the antigen. In yet another exemplary embodiment, a selected, small number of acceptor framework residues can be replaced by the corresponding donor amino acids. The donor framework can be a mature or germline human antibody framework sequence or a consensus sequence. Guidance concerning how to make phenotypically silent amino acid substitutions is provided in Bowie et al., (1990) Science, 247:1306-1310; Cunningham et al., (1989) Science, 244:1081-1085; Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor Laboratory, Cold Spring Harbor, N.Y. (1989); Pearson, (1994) Methods Mol. Biol. 243:307-31; Gonnet et al., (1992) Science 256:1443-45.

The antibody, or antigen-binding portion thereof, can be derivatized or linked to another functional molecule. For example, an antibody can be functionally linked (by chemical coupling, genetic fusion, noncovalent interaction, etc.) to one or more other molecular entities, such as another antibody, a detectable agent, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. One type of derivatized protein is produced by crosslinking two or more proteins (of the same type or of different types). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill. Useful detectable agents with which a protein can be derivatized (or labeled) include fluorescent compounds, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A protein or antibody can also be derivatized with detectable enzymes, such as alkaline phosphatase, horseradish peroxidase, beta-galactosidase, acetylcholinesterase, glucose oxidase and the like. A protein can also be derivatized with a prosthetic group (e.g., streptavidin/biotin and avidin/biotin).

Nucleic acids encoding a functionally active variant of the present antibody or antigen-binding portion thereof are also encompassed by the present invention. These nucleic acid molecules may hybridize with a nucleic acid encoding any of the present antibody or antigen-binding portion thereof under medium stringency, high stringency, or very high stringency conditions. Guidance for performing hybridization reactions can be found in Current Protocols in Molecular Biology, John Wiley & Sons, N.Y. 6.3.1-6.3.6, 1989, which is incorporated herein by reference. Specific hybridization conditions referred to herein are as follows: 1) medium stringency hybridization conditions: 6×SSC at about 45° C., followed by one or more washes in 0.2×SSC, 0.1% SDS at 60° C.; 2) high stringency hybridization conditions: 6×SSC at about 45° C., followed by one or more washes in 0.2×SSC, 0.1% SDS at 65° C.; and 3) very high stringency hybridization conditions: 0.5 M sodium phosphate, 7% SDS at 65° C., followed by one or more washes at 0.2×SSC, 1% SDS at 65° C.

A nucleic acid encoding the present antibody or antigen-binding portion thereof may be introduced into an expression vector that can be expressed in a suitable expression system, followed by isolation or purification of the expressed antibody or antigen-binding portion thereof. Optionally, a nucleic acid encoding the present antibody or antigen-binding portion thereof can be translated in a cell-free translation system (U.S. Pat. No. 4,816,567; Queen et al., (1989) PNAS, 86:10029-10033 (1989).

The present antibodies or antigen-binding portions thereof can be produced by host cells transformed with DNA encoding light and heavy chains (or portions thereof) of the desired antibody. Antibodies can be isolated and purified from these culture supernatants and/or cells using standard techniques. For example, a host cell may be transformed with DNA encoding the light chain, the heavy chain, or both, of an antibody. Recombinant DNA technology may also be used to remove some or all of the DNA encoding either or both of the light and heavy chains that is not necessary for binding, e.g., the constant region.

The present nucleic acids can be expressed in various suitable cells, including prokaryotic and eukaryotic cells, e.g., bacterial cells (e.g., E. coli), yeast cells, plant cells, insect cells, and mammalian cells. A number of mammalian cell lines are known in the art and include immortalized cell lines available from the American Type Culture Collection (ATCC). Non-limiting examples of the cells include all cell lines of mammalian origin or mammalian-like characteristics, including but not limited to, parental cells, derivatives and/or engineered variants of monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells. The engineered variants include, e.g., glycan profile modified and/or site-specific integration site derivatives.

The present invention also provides for cells comprising the nucleic acids described herein. The cells may be a hybridoma or transfectant, such as hybridoma designated as 1E12b.

Alternatively, the present antibody or antigen-binding portion thereof can be synthesized by solid-phase procedures well known in the art. Solid Phase Peptide Synthesis: A Practical Approach by E. Atherton and R. C. Sheppard, published by IRL at Oxford University Press (1989). Methods in Molecular Biology, Vol. 35: Peptide Synthesis Protocols (ed. M. W. Pennington and B. M. Dunn), chapter 7. Solid Phase Peptide Synthesis, 2nd Ed., Pierce Chemical Co., Rockford, Ill. (1984). G. Barany and R. B. Merrifield, The Peptides: Analysis, Synthesis, Biology, editors E. Gross and J. Meienhofer, Vol. 1 and Vol. 2, Academic Press, New York, (1980), pp. 3-254. M. Bodansky, Principles of Peptide Synthesis, Springer-Verlag, Berlin (1984).

The present invention provides for methods for making an antibody or antigen-binding portion thereof that specifically binds to a carbohydrate antigen, (e.g., SSEA-3). For example, a non-human animal is immunized with a composition that includes a carbohydrate antigen (e.g., SSEA-3), and then a specific antibody is isolated from the animal. The method can further include evaluating the binding of the antibody to a carbohydrate antigen.

Any of a variety of carbohydrate antigens, particularly SSEA-3, may be used in the practice of the present invention. Examples of carbohydrate antigens include, but are not limited to Globo antigens such as Globo H, stage-specific embryonic antigen 3 (SSEA-3) (also called Gb5), stage-specific embryonic antigen 4 (SSEA-4), Gb-4 and Gb-3, Lewis antigens such as sLe^{x}, Le^{x}, sLe^{a}, Le^{a} and Le^{y}, polysaccharide antigens such as polysialic acid (PSA), sTn(c) and Tn(c), Thomsen-Friedenreich antigen (TF(c)), the ganglioside such as GD1, GD2, GD3, Fucosyl GM1, GM1, GM2, GM3, GD1α and GM2, sulfitide antigen such as 6Gal-HSO3-SiaLex and 6GluNAc-HSO3-SiaLex. Other carbohydrate antigens include, but are not limited to: α-Galactose, α-Man-6-phosphate, α-L-Rhamnose, α-GalNAc(Tn), α-NeuAc-OCH2C6H4-p-NHCOOCH2, Fucα1-2Galβ1-4GalNAcβ (H types3), NeuAcα2-8NeuAcα, (NeuAcα2-8)2 Polysialic acid, NeuAca2-6Galb, NeuAcb2-6Gala(STn), Galal-3Galbl-4GlaNAcb (NeuAca2-8)3, GalNAcαa-3(Fucα1-2)Galβ (Blood Group A), Galα1-3(Fucal-2)Galβ (Blood Group B), 6Gal-HSO3-SiaLex, 6GluNAc-HSO3-SiaLex and α2-6 sialylated diantennary N-glycans.

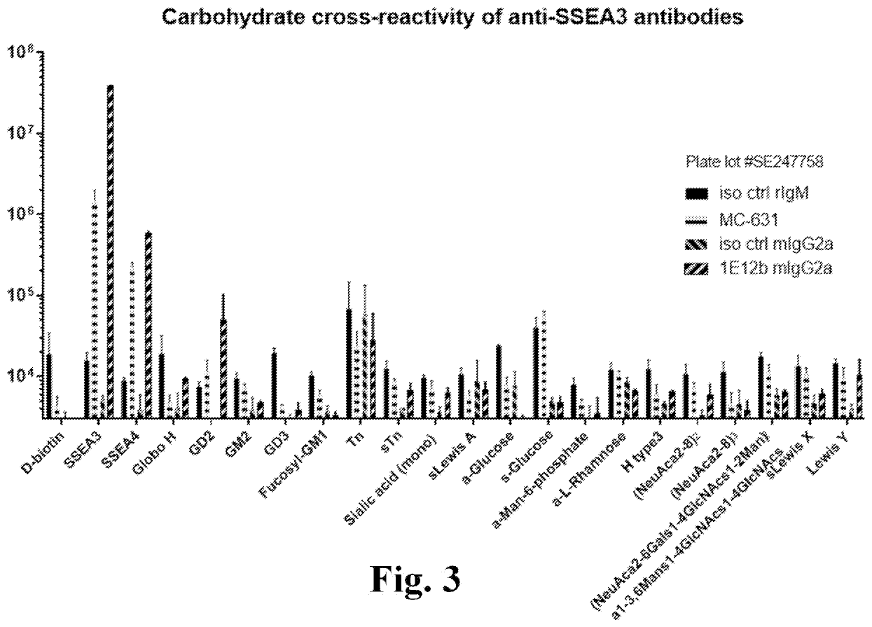

In one embodiment, the anti-SSEA3 antibody or the antigen-binding portion thereof can cross-react or bind with other carbohydrate antigens with high selectivity, as illustrated in FIG. 3. Nonlimiting examples of the carbohydrate antigens are Globo H, SSEA-4, and Lewis antigens.

In one embodiment, the present invention provides a method for making a hybridoma that expresses an antibody that specifically binds to a carbohydrate antigen (e.g., SSEA-3). The method contains the following steps: (a) immunizing an animal with a composition that includes a carbohydrate antigen (e.g., SSEA-3); (b) isolating splenocytes from the animal; (c) generating hybridomas from the splenocytes; and (d) selecting a hybridoma that produces an antibody that specifically binds to SSEA-3. Kohler and Milstein, Nature, 256: 495, 1975. Harlow, E. and Lane, D. Antibodies: A Laboratory Manual, Cold Spring Harbor Laboratory Press, Cold Spring Harbor, N.Y., 1988.

In one embodiment, a carbohydrate antigen is used to immunize mice subcutaneously. One or more boosts may or may not be given. The titers of the antibodies in the plasma can be monitored by, e.g., ELISA (enzyme-linked immunosorbent assay) or flow cytometry. Mice with sufficient titers of anti-carbohydrate antigen antibodies are used for fusions. Mice may or may not be boosted with antigen 3 days before sacrifice and removal of the spleen. The mouse splenocytes are isolated and fused with PEG to a mouse myeloma cell line. The resulting hybridomas are then screened for the production of antigen-specific antibodies. Cells are plated, and then incubated in selective medium. Supernatants from individual wells are then screened by ELISA for human anti-carbohydrate antigen monoclonal antibodies. The antibody secreting hybridomas are replated, screened again, and if still positive for anti-carbohydrate antigen antibodies, can be subcloned by limiting dilution.

Adjuvants that may be used to increase the immunogenicity of one or more of the carbohydrate antigens. Non-limiting examples of adjuvants include aluminum phosphate, aluminum hydroxide, MF59 (4.3% w/v squalene, 0.5% w/v polysorbate 80 (Tween 80), 0.5% w/v sorbitan trioleate (Span 85)), CpG-containing nucleic acid, QS21 (saponin adjuvant), α-Galactosyl-ceramides or synthetic analogs thereof (e.g., C34, see U.S. Pat. No. 8,268,969), MPL (Monophosphoryl Lipid A), 3DMPL (3-O-deacylated MPL), extracts from Aquilla, ISCOMS (Sjolander et al., (1998) J. Leukocyte Biol. 64:713; International Patent Publication Nos. WO1990003184; WO1996011711; WO2000048630; WO1998036772; WO2000041720; WO2006134423 and WO2007026190), LT/CT mutants, poly(D,L-lactide-co-glycolide) (PLG) microparticles, Quil A, interleukins, Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The immunized animal can be any animal that is capable of producing recoverable antibodies when administered an immunogen, such as, but not limited to, rabbits, mice, rats, hamsters, goats, horses, monkeys, baboons, and humans. In one aspect, the host is transgenic and produces human antibodies, e.g., a mouse expressing the human immunoglobulin gene segments (U.S. Pat. Nos. 8,236,311; 7,625,559 and 5,770,429; Lonberg et al., (1994) Nature 368(6474):856-859; Lonberg, N., Handbook of Experimental Pharmacology 113:49-101 (1994); Lonberg, N. and Huszar, D., (1995) Intern. Rev. Immunol., 13:65-93; Harding, F. and Lonberg, N., (1995) Ann. N.Y. Acad. Sci., 764:536-546).

After the host is immunized and the antibodies are produced, the antibodies are assayed to confirm that they are specific for the antigen of interest and to determine whether they exhibit any cross-reactivity with other antigens. One method of conducting such assays is a sera screen assay as described in U.S. Patent Publication No. 2004/0126829. Anti-carbohydrate antigen antibodies can be characterized for binding to the carbohydrate by a variety of known techniques. For example, in an ELISA, microtiter plates are coated with the toxin or toxoid antigen in PBS and then blocked with irrelevant proteins such as bovine serum albumin (BSA) diluted in PBS. Dilutions of plasma from toxin-immunized mice are added to each well and incubated. The plates are washed and then incubated with a secondary antibody conjugated to an enzyme (e.g., alkaline phosphatase). After washing, the plates are developed with the enzyme's substrate (e.g., ABTS), and analyzed at a specific OD. In other embodiments, to determine if the selected monoclonal antibodies bind to the target carbohydrate antigen or epitopes, the antibody can be biotinylated which can then be detected with a streptavidin labeled probe. Anti-carbohydrate antigen antibodies can be tested for reactivity with the carbohydrate by Western blotting.

Hybridomas that produce antibodies that bind, preferably with high affinity, to the carbohydrate antigen, can then be subcloned and further characterized. One clone from each hybridoma, which retains the reactivity of the parent cells (by ELISA), can then be chosen for making a cell bank, and for antibody purification.

Antibodies, or antigen-binding fragments, variants or derivatives thereof of the present disclosure can also be described or specified in terms of their binding affinity to an antigen. The affinity of an antibody for a carbohydrate antigen can be determined experimentally using any suitable method (see, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-carbohydrate antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer.

The present antibodies or antigen-binding portions thereof have in vitro and in vivo therapeutic, prophylactic, and/or diagnostic utilities. For example, these antibodies can be administered to cells in culture, e.g., in vitro or ex vivo, or to a subject, e.g., in vivo, to treat, inhibit, prevent relapse, and/or diagnose cancer.

The antibodies or antigen-binding portions thereof can be used on cells in culture, e.g., in vitro or ex vivo. For example, cells can be cultured in vitro in culture medium and contacted by the anti-SSEA3 antibody or fragment thereof. The methods can be performed on cells present in a subject, as part of an in vivo (e.g., therapeutic or prophylactic) protocol. For in vivo embodiments, the contacting step is effected in a subject and includes administering an anti-toxin antibody or portion thereof to the subject under conditions effective to permit binding of the antibody or portion thereof, to a carbohydrate antigen (e.g., SSEA-3) expressed on one or more cancer cells in the subject, e.g., in the breast cancer cell.

The antibody or antigen-binding portion thereof can be administered alone or in combination with another therapeutic agent, e.g., a second monoclonal or polyclonal antibody or antigen-binding portion thereof or a chemotherapeutic agent. The combination product may be a mixture of the two compounds or they may be covalently attached. In one example, the antibody or antigen-binding portion thereof specifically binds to Globo H is combined with an antibody (monoclonal or polyclonal) or antigen-binding portion thereof specifically binds VEGF. In another example, the second agent is a chemotherapy agent (e.g., cyclophosphamide, 5-fluorouracil or Actinomycin-D). The antibodies can also be administered in combinations with a cancer vaccine, e.g., Globo H conjugated with Diphtheria Toxin and a saponin adjuvant.

### Methods for Inhibiting Cancer Cells

The invention also provides methods for inhibiting the growth of a cell in vitro, ex vivo or in vivo, wherein the cell, such as a cancer cell, is contacted with an effective amount of an antibody or an antigen-binding portion thereof as described herein. Pathological cells or tissue such as hyperproliferative cells or tissue may be treated by contacting the cells or tissue with an effective amount of an antibody or an antigen-binding portion thereof of this invention. The cells, such as cancer cells, can be primary cancer cells or can be cultured cells available from tissue banks such as the American Type Culture Collection (ATCC). The pathological cells can be cells of an SSEA-3 expressing cancer, gliomas, meningiomas, pituitary adenomas, or a CNS metastasis from systemic cancer, lung cancer, prostate cancer, breast cancer, hematopoietic cancer or ovarian cancer. The cells can be from a vertebrate, preferably a mammal, more preferably a human (U.S. Patent Publication No. 20040087651; Balassiano et al., (2002) Intern. J. Mol. Med. 10:785-788; Thorne, et al., (2004) Neuroscience 127:481-496; Fernandes, et al., (2005) Oncology Reports 13:943-947; Da Fonseca, et al., (2008) Surgical Neurology 70:259267; Da Fonseca, et al., (2008) Arch. Inmunol. Ther. Exp. 56:267-276; Hashizume, et al., (2008) Neuroncology 10:112-120). In one embodiment, the cancer is Globo H expressing cancer. In another embodiment, the cancer is SSEA-3 expressing cancer. In yet another embodiment, the cancer is SSEA-4 expressing cancer. Globo H expressing cancer, SSEA-3 expressing cancer and SSEA-4 expressing cancer include, but are not limited to, breast cancer, lung cancer, prostate cancer, pancreatic cancer, gastric cancer, ovarian cancer and endometrial cancer and colon cancer, liver cancer, nasopharyngeal cancer, skin cancer, oral cancer, renal cancer, brain cancer, cervical cancer and bladder cancer.

In vitro efficacy of the present antibody or the antigen-binding portion thereof can be determined using methods well known in the art. For example, the cytotoxicity of the antibody or the antigen-binding portion thereof may be studied by MTT [3-(4,5-dimethylthiazol-2-yl)-2,5-diphenyl tetrazolium bromide] cytotoxicity assay. MTT assay is based on the principle of uptake of MTT, a tetrazolium salt, by metabolically active cells where it is metabolized into a blue colored formazon product, which can be read spectrometrically. J. of Immunological Methods 65: 55 63, 1983. The cytotoxicity of the present antibody or the antigen-binding portion thereof may be studied by colony formation assay. Functional assays for binding Globo H antigen may be performed via ELISA. Cell cycle block by the antibody or the antigen-binding thereof may be studied by standard propidium iodide (PI) staining and flow cytometry. Invasion inhibition may be studied by Boyden chambers. In this assay a layer of reconstituted basement membrane, Matrigel, is coated onto chemotaxis filters and acts as a barrier to the migration of cells in the Boyden chambers. Only cells with invasive capacity can cross the Matrigel barrier. Other assays include, but are not limited to cell viability assays, apoptosis assays, and morphological assays.

Assays can also be done in vivo using a murine model. See, e.g., B. Teicher, Tumor Models for Efficacy Determination. Mol Cancer Ther 2006; 5: 2435-2443.”

### Pharmaceutical Composition

In one embodiment, the present invention provides pharmaceutical compositions comprising an antibody or antigen-binding portion thereof described herein, and a pharmaceutically acceptable carrier. In another embodiment, the pharmaceutical composition comprises an isolated nucleic acid encoding the present antibody or antigen-binding portion thereof, and a pharmaceutically acceptable carrier. Pharmaceutically acceptable carriers include any and all solvents, dispersion media, isotonic and absorption delaying agents, and the like that are physiologically compatible. In one embodiment, the composition is effective to inhibit cancer cells in a subject.

Routes of administration of the present pharmaceutical compositions include, but are not limited to, intravenous, intramuscular, intransal, subcutaneous, oral, topical, subcutaneous, intradermal, transdermal, subdermal, parenteral, rectal, spinal, or epidermal administration.

The pharmaceutical compositions of the present invention can be prepared as injectables, either as liquid solutions or suspensions or as solid forms which are suitable for solution or suspension in liquid vehicles prior to injection. The pharmaceutical composition can also be prepared in solid form, emulsified or the active ingredient encapsulated in liposome vehicles or other particulate carriers used for sustained delivery. For example, the pharmaceutical composition can be in the form of an oil emulsion, water-in-oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the pharmaceutical composition.

The present antibodies or antigen-binding portions thereof are formulated into pharmaceutical compositions for delivery to a mammalian subject. The pharmaceutical composition is administered alone, and/or mixed with a pharmaceutically acceptable vehicle, excipient or carrier. Suitable vehicles are, for example, water, saline, dextrose, glycerol, ethanol, or the like, and combinations thereof. In addition, the vehicle can contain minor amounts of auxiliary substances such as wetting or emulsifying agents, pH buffering agents, or adjuvants. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, detergents, liposomal carriers, or excipients or other stabilizers and/or buffers. Other physiologically acceptable compounds include wetting agents, emulsifying agents, dispersing agents or preservatives. See e.g., the 21st edition of Remington's Pharmaceutical Science, Mack Publishing Company, Easton, Pa. (“Remington's”). The pharmaceutical compositions of the present invention can also include ancillary substances, such as pharmacological agents, cytokines, or other biological response modifiers.

Furthermore, the pharmaceutical compositions can be formulated into pharmaceutical compositions in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Actual methods of preparing such dosage forms are known or will be apparent, to those skilled in the art. See, e.g., Remington's Pharmaceutical Sciences, Mack Publishing Company, Easton, Pa., 21st edition.

Pharmaceutical compositions can be administered in a single dose treatment or in multiple-dose treatments on a schedule and over a time period appropriate to the age, weight, and condition of the subject, the particular composition used, and the route of administration, whether the pharmaceutical composition is used for prophylactic or curative purposes, etc. For example, in one embodiment, the pharmaceutical composition according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an antibody according to the invention, e.g., the period of time over which the pharmaceutical composition is administered, can vary, depending on any of a variety of factors, e.g., subject response, etc. For example, the pharmaceutical composition can be administered over a period of time ranging from about one or more seconds to one or more hours, one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

For ease of administration and uniformity of dosage, oral or parenteral pharmaceutical compositions in dosage unit form may be used. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The data obtained from the cell culture assays and animal studies can be used in formulating a range of dosage for use in humans. In one embodiment, the dosage of such compounds lies within a range of circulating concentrations that include the ED₅₀ with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture (Sonderstrup, Springer, Sem, (2003) Immunopathol. 25: 35-45; Nikula et al., (2000) Inhal. Toxicol. 4(12):123-53).

An exemplary, non-limiting range for a therapeutically or prophylactically effective amount of an antibody or antigen-binding portion of the invention is from about 0.001 to about 60 mg/kg body weight, about 0.01 to about 30 mg/kg body weight, about 0.01 to about 25 mg/kg body weight, about 0.5 to about 25 mg/kg body weight, about 0.1 to about 20 mg/kg body weight, about 10 to about 20 mg/kg body weight, about 0.75 to about 10 mg/kg body weight, about 1 to about 10 mg/kg body weight, about 2 to about 9 mg/kg body weight, about 1 to about 2 mg/kg body weight, about 3 to about 8 mg/kg body weight, about 4 to about 7 mg/kg body weight, about 5 to about 6 mg/kg body weight, about 8 to about 13 mg/kg body weight, about 8.3 to about 12.5 mg/kg body weight, about 4 to about 6 mg/kg body weight, about 4.2 to about 6.3 mg/kg body weight, about 1.6 to about 2.5 mg/kg body weight, about 2 to about 3 mg/kg body weight, or about 10 mg/kg body weight.

The pharmaceutical composition is formulated to contain an effective amount of the present antibody or antigen-binding portion thereof, wherein the amount depends on the animal to be treated and the condition to be treated. In one embodiment, the present antibody or antigen-binding portion thereof is administered at a dose ranging from about 0.01 mg to about 10 g, from about 0.1 mg to about 9 g, from about 1 mg to about 8 g, from about 2 mg to about 7 g, from about 3 mg to about 6 g, from about 10 mg to about 5 g, from about 20 mg to about 1 g, from about 50 mg to about 800 mg, from about 100 mg to about 500 mg, from about 0.01 μg to about 10 g, from about 0.05 μg to about 1.5 mg, from about 10 μg to about 1 mg protein, from about 30 μg to about 500 μg, from about 40 μg to about 300 μg, from about 0.1 μg to about 200 μg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy and can be determined by one of ordinary skill in the art without undue experimentation.

The present antibodies, antigen-binding portions thereof, pharmaceutical compositions and methods can be used in all vertebrates, e.g., mammals and non-mammals, including human, mice, rats, guinea pigs, hamsters, dogs, cats, cows, horses, goats, sheep, pigs, monkeys, apes, gorillas, chimpanzees, rabbits, ducks, geese, chickens, amphibians, reptiles and other animals.

The following examples of specific aspects for carrying out the present invention are offered for illustrative purposes only and are not intended to limit the scope of the present invention in any way.

### EXAMPLES

### Example 1: Hybridoma Fusion and Screening

A classical hybridoma fusion was performed. Balb/c mice received their first immunization with 0.2 and 2.0 μg SSEA3-KLH (Keyhole Limpet Hemocyanin) vaccine plus 20 μg OBI-821 saponin adjuvant and eight subsequent boosters on days 0, 7, 14, 21, 28, 35, 42 and 49 via SC injection. Bleedings trials were performed on day 10, 17, 24, 31, 38, 45, and 52, and the serum was tested to check for titers of anti-SSEA3 antibody. Two mice were found to produce high anti-SSEA3 IgG and IgM titers (2560×) and were used for hybridoma production. Mouse myeloma cells were used for fusion with the mouse splenocytes following procedure of a KÖhler and Milstein (KOhler G. and Milstein C, 1975). Hybridoma supernatants were screened by affinity ELISA and using anti-SSEA3 MC-631 mAb (BioLegend, Cat No. 330302) as positive controls. The OD of hybridoma clone with no dilution of supernatant>background×2 was selected. The hybridoma clone 1E12b was selected.

### Example 2: ELISA Binding Assay

Reagent

Plating Antigen: SSEA3-ceramide stock (OBI Pharma, Inc.) powder was dissolved in methanol/CHCl₃ solution (methanol: CHCl₃: H₂O=50:8:25) at −20° C. Primary Antibody: serum sample or Rat anti-SSEA3 IgM antibody (BioLegend, Cat. No. 330302).

### Secondary Antibody:

1. Goat anti-mouse IgG-AP (Southern Biotech, Cat. No. 1030-04)
2. Goat anti-mouse IgM-AP (Southern Biotech, Cat. No. 1020-04)
3. Goat anti-rat IgM-AP (Jackson Immuno Research, Cat. No. 112-055-075)
   Blocking Buffer (Sigma, Cat. No. B6429)
   Substrate Solution: Alkaline Phosphatase Yellow (pNPP) Liquid Substrate System for ELISA (Sigma, Cat. No. P7998)
   Stop Solution: Alkaline Phosphatase Stop Solution (Sigma, Cat. No. A5852)

Procedure

### Reagent Preparation

1. Store SSEA3-ceramide powder dissolved in methanol/CHCl₃ solution (methanol: CHCl₃: H₂O=50:8:25) at −20° C. as stock (the concentration of SSEA3-ceramide stock is labeled on the glass bottle). Then, aliquot the stock to other glass bottles for using and store them at 4° C. refrigerator.
2. 10× Blocking Buffer: dilute the 10× Blocking Buffer in double-distilled water (d.d. H₂O) to 1× for use.
3. Wash Buffer: add 0.5 mL of Tween 20 into 1 L of PBS to make a 0.05% Tween 20 in PBS.
4. Secondary antibody: Use the following procedure to make a final concentration of 0.3 mg/mL antibody. Rehydrate with 1.0 mL d.d. H₂O. Add 1.0 mL of glycerol (ACS grade or better) to a 2 mL Eppendorf to reach the 1 mL scale tick of the Eppendorf. Transfer the 0.5 mL of rehydrated antibody into the glycerol. Mix well and then transfer the 1.5 mL of antibody back to the bottle of 0.5 mL antibody. Mix well, aliquot, and store at −20+2° C. refrigerator.

### Antigen Coating

1. For each 96-well plate: add 20 μg of SSEA3-ceramide to ethanol to a total of 5 mL coating buffer and mix gently. (0.2 μg of SSEA3-ceramide/well×100-well plate=20 μg; 50 μL/well×100 well=5 mL)
2. Pipet 50 μL of coating SSEA3-ceramide into each well on ice. Label, cover with a lid, and incubate room temperature overnight.
3. Pipet into each well 100 μL of Blocking Buffer and incubate at room temperature (22-26° C.) for approximately 0.5 hours.
4. Prepare a 96-well “Dilution plate,” label in the space above the top row of the sample's ID.
5. Pipet 144 μL of 1× Blocking Buffer into one Eppendorf.
6. Pipet 6 μL of the positive control [anti-SSEA3 antibody (stock: 0.5 mg/mL)] into the eppendorf for producing a 1:25 dilution.
7. For standard, add 120 μL 1:25 diluted anti-SSEA3 antibody to the top well of column 2. Then add 180 μL Blocking buffer to the rest wells of column 2, leaving the first column (column 1) empty for blank.
8. For hybridoma clone supernatant samples, add 120 μL original positive clone supernatant to the top well of column 3-12. For serum samples, add 4.8 μL serum samples and 115.2 μL Blocking buffer into the top of column 3-12 to make a 1:25 diluted serum sample. Then add 60 μL Blocking buffer to the rest wells of column 3-12.
9. Mix well by pipetting repeatedly.
10. Transfer 60 μL of the diluted anti-SSEA3 antibody and samples from the first well to the second well.
11. Mix well by pipetting repeatedly.
12. For standard, the anti-SSEA3 antibody dilutions in column 2 as below:
   1^{st} well=1:25 (20000 ng/mL)
   2^{nd} well=1:100 (5000 ng/mL)
   3^{rd} well=1:400 (1250 ng/mL)
   4^{th} well=1:1600 (312.5 ng/mL)
   5^{th} well=1:6400 (78.125 ng/mL)
   6^{th} well=1:25600 (19.53 ng/mL)
   7^{th} well=1:102400 (4.88 ng/mL)
   8^{th} well=1:409600 (2.11 ng/mL)
   Anti-SSEA3 antibody: 1:25 (four-fold dilution)
13. For hybridoma supernatant samples, the dilutions are made as below:
   1^{st} well=1:1
   2^{nd} well=1:2
   3^{rd} well=1:4
   4^{th} well=1:8
   5^{th} well=1:16
   6^{th} well=1:32
   7^{th} well=1:64
   8^{th} well=1:128
   Hybridoma clone supernatant: 1:1(two-fold dilution)
14. For serum samples, the dilutions are made as below:
   1^{st} well=1:25
   2^{nd} well=1:50
   3^{rd} well=1:100
   4^{th} well=1:200
   5^{th} well=1:400
   6^{th} well=1:800
   7^{th} well=1:1600
   8^{th} well=1:3200
   Serum sample: 1:25 (two-fold dilution)

### Sample Adding

### Addition of Primary Antibody to Test Plate.

1. After incubation with Blocking Buffer, remove the Blocking solution by aspiration and wash each well 3 times with 200 μL Wash Buffer.
2. Use a pipetman to add 50 μL diluted positive clone supernatant and anti-SSEA3 antibody from Dilution Plate to wells in the Test Plate.
3. Cover, label, and incubate “Test Plate” at room temperature for approximately 1 hour.
4. After incubation of the plates, aspirate all wells and then wash all wells three times with 200 μL Wash Buffer.

### Addition of Secondary Antibody to the Test Plate.

5. For one 96-well plate: add 25 μL of Secondary Antibody [Goat anti-rat IgM-AP for Standard anti-SSEA3 antibody and Goat anti-mouse IgM or IgG-AP for hybridoma supernatant] to 4975 μL of Blocking Buffer (1:200) and mix gently. (50 μL/well×100-well plate=5 mL)
6. Pipet 50 μL of Secondary Antibody Solution into each well. Cover, label, and incubate at room temperature for approximately 45 minutes.
7. After incubation is complete, aspirate Secondary Antibody Solution from all wells and wash all wells four times with 200 μL Wash Buffer.

### Addition of Substrate Solution to Test Plate.

8. Pipet 100 μL Substrate Solution into each well and incubate for 20 minutes at 37±2° C.
9. Stop by the addition of 50 μL of Stop Solution, mix well, and then read plate at 405 nm on the ELISA Plate Reader.

### Data Analysis

1. The endpoint titer of each column is defined as the reciprocal of the highest dilution that gives a reading above the cutoff value.
2. Subtract sample OD value in the Test Sample Dilution Plate with the OD value of the same sample in the Subtraction Plate.
3. Controls are high QC and low QC as positive control and secondary antibody only as a negative control.
4. Cutoff value: X+0.1. X is the mean OD value of negative control.
5. Record the layout of positive control, negative control, and samples and plate reader printouts, calculations, etc. in the lab notebook.
6. Data are analyzed statistically by the Mann-Whitney test using GraphPad Prism 5 Software.

Result

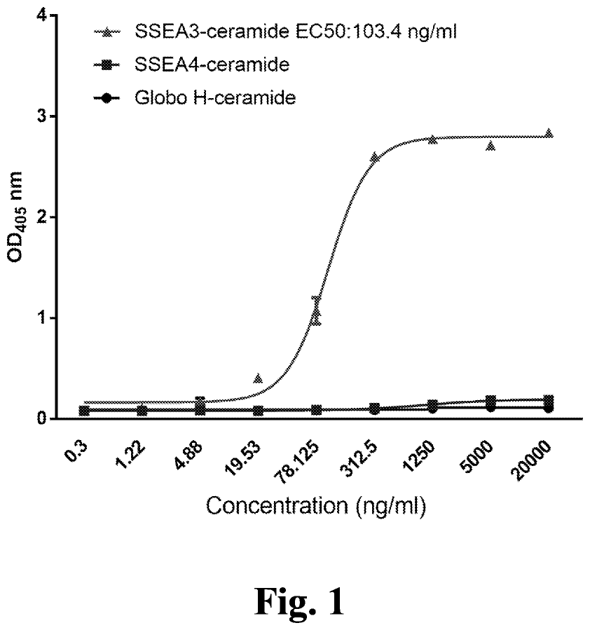

FIG. 1 indicated the binding affinity of 1E12b to SSEA-3 ceramide is 5.1E-08 by ELISA. In addition, there was no binding to SSEA-4 ceramide and Globo H ceramide.

### Example 3: Cell Binding Assay

Reagent

0.05% Trypsin (Invitrogen, Cat No. 25300054): store at 4° C.
Trypan Blue Solution, 0.4% in Phosphate Buffered Saline (Hyclone, Cat No. SV30084.01)
FACS buffer: 1% BSA and 0.1% Sodium azide in PBS:
1. BSA (Sigma, Cat No. SI-A4503-100G) Sodium azide (Sigma, Cat No. S2002-25 g)
2. PBS (Gibco, Cat No. 70011-044-500 mL)

### Primary Antibody:

1. 1E12b
2. Isotype control antibody: Human IgG Fc (Southern Biotech, Cat No. 0160-01) Secondary Antibody: anti-human IgG (Fc specific)-FITC (Sigma, Cat No. F9512)

Procedure

### Cell Culture

1. MCF-7 cell (Globo H-high/SSEA4-high/SSEA3- high) were cultured in Minimum essential medium (Invitrogen, Cat No. 10370021) with 2 mM L-glutamine (Invitrogen, Cat No. 25030081) and 1 mM sodium pyruvate (Invitrogen, Cat No. 11360070) and supplemented with 0.01 mg/mL insulin (Sigma, Cat No. SI-I9278), 10% fetal bovine serum (Invitrogen, Cat No. 16000044).
2. SKOV3 cell (Globo H-low/SSEA4-high/SSEA3-high) were cultured in McCoy's 5a Medium Modified (Invitrogen, Cat No. 166600082) with 10% fetal bovine serum (Invitrogen, Cat No. 16000044).
3. SKBR3 cell (Globo H-low/SSEA4-low/SSEA3-high) were cultured in McCoy's 5a Medium Modified (Invitrogen, Cat No. 166600082) with 10% fetal bovine serum (Invitrogen, Cat No. 16000044).

### Cell Staining

### Suspend Test Cells

1. In the case of monolayer cells, discard media from test sample flask. Rinse the cell with 5 mL PBS twice. Add 1 mL of 0.05% trypsin to a flask, swirl to cover all surface area, and put in 37° C. CO2 incubator for 5-10 minutes.
2. Add 5 mL of complete growth medium and aspirate cells by gently pipetting.
3. Transfer cell suspension to a 15 mL conical tube.
4. Centrifuge the tube for 5 minutes at 200 g.
5. Following centrifuged, decant the supernatant and agitate the cell pellet. Count Test Cells
1. Add 1-2 mL FACS buffer to the cell pellet and mix well by pipetting.
2. Add cell onto the 5 mL Polystyrene Round-Bottom Tube with Cell-Strainer Cap (Falcon, Cat No. 352235) to obtain intact and viable single cells.
3. Transfer 10 μL cell suspension to a microcentrifuge tube.
4. Add 10 μL of Trypan Blue Solution (0.4% Trypan blue) and mix well by pipetting.
5. Count viable cells on a hematocytometer.
6. Adjust cell concentration to be 4×10⁶ cells/mL, and then pipet 50 μL cell suspension into polystyrene round tube to make 2×10⁵ cells per tube.

### Addition of the First Antibody

1. Make an antibody diluent with 10 μg/mL.
2. Add 50 μL antibody diluent to 50 μL cell suspension (6.2.3.6) to reach 0.5 μg antibody per tube.
3. Vortex gently each tube to mix cell suspension and first antibody well.
4. Place tubes on ice for incubation of approximately 0.5 hr.
5. Fill every tube with 1 mL FACS buffer and wash one time each by centrifuging at 400 g for 5 minutes.
6. Remove the supernatant with vacuum suction. Be careful the suction action and avoid suction tip touching the bottom of the tube that may cause the cell loss.

### Addition of Secondary Antibody

1. Dilute Secondary antibody in FACS buffer to a final antibody concentration of 4 μg/mL. ※Dilute Secondary antibody with 1:100 dilution for anti-human IgG (Fc specific)-FITC (Sigma, Cat No. F9512).
2. Add 100 μL diluted secondary antibody to every tube.
3. Vortex gently each tube to mix cell suspension and secondary antibody well.
4. Place tubes on ice and avoid light for incubation of approximately 0.5 hr.
5. Fill every tube with 1 mL FACS buffer and wash one time each by centrifuging at 400 g for 5 minutes.
6. Remove the supernatant with vacuum suction. Be careful the suction action and avoid suction tip touching the bottom of the tube that may cause the cell loss.
7. Add 300 μL of 4% paraformaldehyde-fixed solution to every tube.
8. Place tubes on ice and avoid light for incubation of approximately 0.5 hr.
9. Fill every tube with 1 mL FACS buffer and wash one time each by centrifuging at 400 g for 5 minutes.
10. Resuspend test cell tube with 400 μL of FACS buffer.
11. Store test tube in 4±2° C. refrigerator and avoid light.

### Flow Cytometry Analysis

1. Perform flow cytometry right after staining. Or perform flow cytometry within one week if the cells fixed by 4% paraformaldehyde buffer.
2. The percentage of cell binding was analyzed by FCS Express 4 Flow Research software. In the histogram plot, isotype control was gated and defined that 5% of gated cells were background. Based on the setting of the background, the percentage of binding region (M) of test cell tube was determined. In this setting, test cells of 5% or greater above the isotype control are considered as positive binding of cells.

Result

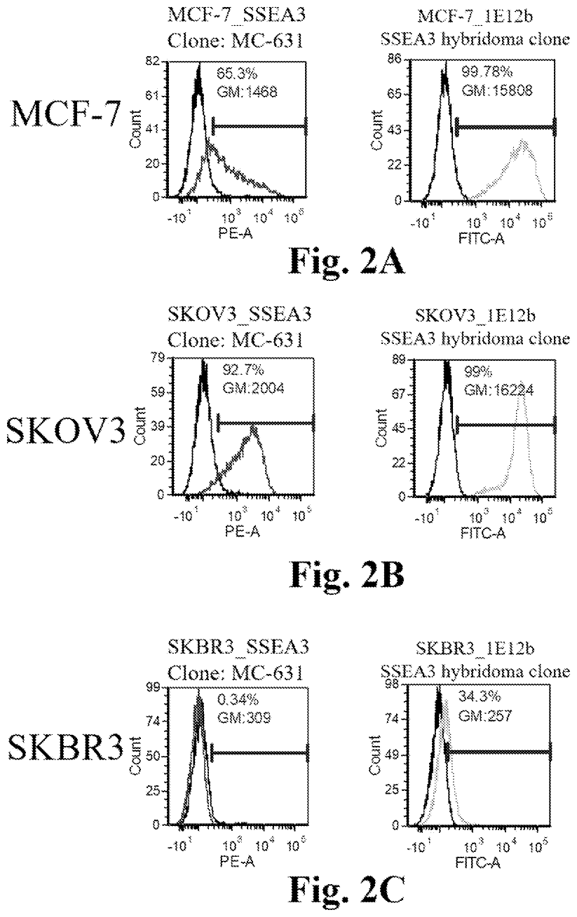

FIG. 2 indicated the 1E12b has a strong binding affinity to high SSEA-3 expression cell line, MCF-7 (FIG. 2A) and SKOV3 (FIG. 2B) cells. It is no binding to low SSEA3 expression cell line, SKBR3 (FIG. 2C) cell.

### Example 4: Cross-Reactivity of Tumor-Associated Sugars with Anti-SSEA3 Antibody

Reagent

Blocking Buffer: SuperBlock (PBS) blocking buffer (ThermoFisher, Cat No. 37515) ELISA Washing Buffer: 0.05% Tween 20 (Sigma, Cat No. P2287-500 mL)/PBS: (Sigma, Cat No. P5493-4 L)
ELISA substrate: SuperSignal ELISA Femto Maximum Sensitivity Substrate (ThermoSci, Cat No. PIE37074)

### Primary Antibody:

1. Anti-SSEA3 antibody (MC-631: BioLegend, Cat No. 330302)
2. 1E12b mIgG2a 04MM-180320
3. mIgG2a isotype control (BioLegend, Cat No. 401502)
4. rIgM isotype control (eBiosecince #14-4341-85)
   All antibodies were diluted in SuperBlock at 5 μg/mL.

### Secondary Antibody:

1. Goat anti-mouse IgG-HPR (JacksonImmRes, Cat No. 109-035-003)
2. Goat anti-rat IgM-HRP (JacksonImmRes, Cat No. 112-035-020)
All antibodies were stored with 1:50,000 dilution in SuperBlock
Each biotinylated sugar listed in Table 2 and D-Biotin (Carbosynth, Cat No. FB02633) was dissolved in 1×PBS to make a 1 mg/mL solution.

TABLE 2
List of Tumor-Associated Sugar

| | Manufacturer/ |
|---|---|
| Sugar | Cat No. |
| α-Glucose | NCKU |
| α-Galactose | NCKU |
| α-Man-6-phosphate | NCKU |
| α-L-Rhamnose | NCKU |
| H types3: Fucα1-2Galβ1-4GalNAcβ | NCKU |
| (NeuAcα2-8)₂ | NCKU |
| NeuAcβ2-6GalNAcα | NCKU |
| (NeuAcα2-8)₃ | NCKU |
| Blood Group B: Galα1-3(Fucα1-2)Galβ | NCKU |
| 6Gal-HSO₃-SiaLex: | NCKU |
| Neu5Acα2-3(6-HSO₃)Galβ1-4(Fucα1-3)GlcNAcβ | |
| 6GlcNAc-HSO₃-SiaLex: | NCKU |
| Neu5Acα2-3Galβ1-4(Fucα1-3)(6-HSO₃)GlcNAcβ | |
| α2-6 sialylated diantennary N-glycans: | NCKU |
| (NeuAcα2-6Galα1-4GlcNAcα1-2Man)₂α1-3,6Manα1- | |
| 4GlcNAcα1-4GlcNAc | |
| GD2: GalNAcβ1-4(NeuAcα2-8NeuAcα2-3)Galβ1-4Glc-β | OligoTech |
| | Cat No. |
| | GLY094-NAc- |
| | sp3-Bt |
| GM2: GalNAcβ1-4(NeuAcα2-3)Galβ1-4Glc-β | OligoTech |
| | Cat No. |
| | GLY093-NAc- |
| | sp3-Bt |
| SSEA4 hexaose: | OligoTech |
| Neu5Acα2-3Galβ1-3GalNAcβ1-3Galα1-4Galβ1-4Glcβ | Cat# GLY131- |
| | NAc-sp3-Bt |
| GD3: NeuAcα2-8NeuAcα2-3Galβ1-4Glc-β | OligoTech |
| | Cat No. |
| | GLY091-NAc- |
| | sp3-Bt |
| Fucosyl-GM1: | OligoTech |
| Fucα1-2Galβ1-3GalNAcβ1-4(Neu5Acα2-3)Galβ1-4Glcβ | Cat No. |
| | GLY103-NAc- |
| | sp3-Bt |
| Globo H: Fucα1-2Galβ1-3GalNAcβ1-3Galα1-4Galβ1-4Glcβ | OBI Pharma, Inc |
| SSEA3: Galβ1-3GalNAcβ1-3Galα1-4Galβ1-4Glcβ | OBI Pharma, Inc |
| SSEA4: Neu5Acα2-3Galβ1-3GalNAcβ1-3Galα1-4Galβ1-4Glcβ | OBI Pharma, Inc |
| Tn: α-GalNAc | GlycoTech |
| | Cat No. 01-010 |
| sTn: NeuAcα2-6GalNAcα | GlycoTech |
| | Cat No. 01-059 |
| Sialic acid (mono): α-Neu5Ac | GlycoTech |
| | Cat No. 01-012 |
| sLewis A: NeuAcα2-3Galβ1-3(Fucα1-4)GlcNAcβ | GlycoTech |
| | Cat No. 01-044 |
| sLewis X: NeuAcα2-3Galβ1-4(Fucα1-3)GlcNAcβ | GlycoTech, |
| | Cat No. 01-045 |
| Lewis Y: Fucα1-2Galβ1-4(Fucα1-3)GlcNAcβ | GlycoTech |
| | Cat No. 01-043 |

*NCKU: National Cheng-Kung University (Taiwan).
Materials supplied by NCKU/OBI Pharma are not from a commercial source.

Procedure

1. Wash plates with ELISA washing buffer 200 μL/well.
2. Dilute the biotinylated sugars stock by PBS at 1 gig/mL, add 50 μL diluted biotinylated sugars into the Coat plates to make 50 ng/well biotinylated sugars and incubate at room temperature for 2 hours.
3. Wash plates once with ELISA washing buffer 200 μl/well.

Result

FIG. 3 indicated the carbohydrate cross-reactivity ELISA system with biotin-labeled sugars, both 1E12b, and MC-631 (commercial anti-SSEA3 Ab) could bind to SSEA-3 and SSEA-4 antigens.

While specific aspects of the invention have been described and illustrated, such aspects should be considered illustrative of the invention only and not as limiting the invention as construed in accordance with the accompanying claims. All publications and patent applications cited in this specification are herein incorporated by reference in their entirety for all purposes as if each individual publication or patent application were specifically and individually indicated to be incorporated by reference in its entirety for all purposes. Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed is:

1. An antibody, or an antigen-binding portion thereof, comprising:
a heavy chain variable domain comprising an amino acid sequence with 80-100% identity to SEQ ID NO: 3; and
a light chain variable domain comprising an amino acid sequence with 80-100% identity to SEQ ID NO: 4.

2. The antibody, or an antigen-binding portion thereof of claim 1, further comprising:
a first heavy chain complementarity determining region (HCDR1) having an amino acid sequence with 90-100% identity to SEQ ID NO: 5;
a second heavy chain complementarity determining region (HCDR2) having an amino acid sequence with 90-100% identity to SEQ ID NO: 6;
a third heavy chain complementarity determining region (HCDR3) having an amino acid sequence with 90-100% identity to SEQ ID NO: 7;
a first light chain complementarity determining region (LCDR1) having an amino acid sequence with 90-100% identity to SEQ ID NO: 8;
a second light chain complementarity determining region (LCDR2) having an amino acid sequence with 90-100% identity to SEQ ID NO: 9; and
a third light chain complementarity determining region (LCDR3) having an amino acid sequence with 90-100% identity to SEQ ID NO: 10.

3. An antibody, or an antigen-binding portion thereof, produced by a hybridoma designated as 1E12b deposited under ATCC Accession Number PTA-126149.

4. The antibody or antigen-binding portion thereof of claim 1, wherein the variable domain is capable of binding a carbohydrate antigen.

5. The antibody or antigen-binding portion thereof of claim 4, wherein the carbohydrate antigen is a stage-specific embryonic antigen 3 (SSEA-3) or a stage-specific embryonic antigen 4 (SSEA-4).

6. The antibody or antigen-binding portion thereof of claim 1, wherein the antibody or antigen-binding portion thereof is selected from the group consisting of a whole immunoglobulin molecule, an scFv, a Fab fragment, an F(ab′)2, and a disulfide linked Fv.

7. The antibody, or an antigen-binding portion thereof of claim 1, wherein the antibody is a humanized antibody.

8. The antibody, or an antigen-binding portion thereof of claim 7, wherein the antibody is an IgG or an IgM.

9. The antibody, or an antigen-binding portion thereof of claim 7, wherein the antibody further comprises a chimeric antigen receptor (CAR) domain specific for SSEA-3.

10. The antibody, or an antigen-binding portion thereof of claim 9, wherein the CAR domain comprises a transmembrane domain and a intracellular signaling domain, wherein the transmembrane domain comprises a transmembrane domains of CD8 and/or CD28, and the intracellular signaling domain comprises an intracellular signaling domains selected from the groups consisting of CD27, CD28, CD137, OX40, ICOS, CD3zeta and any combination thereof.

11. A bi-specific antibody or antigen-binding portion thereof, comprising:
the antibody, or the antigen-binding portion thereof of claim 1, wherein the antibody, or the antigen-binding portion thereof further comprises a first binding domain that specifically binds to Globo series antigens and a second binding domain that specifically binds to T cell surface antigens.

12. The bi-specific antibody or antigen-binding portion of claim 11, wherein the Globo series antigens comprising Globo H, SSEA-3 or SSEA-4.

13. The bi-specific antibody or antigen-binding portion of claim 11, wherein the T cell surface antigens comprise CD2, CD3, CD4, CD5, CD6, CD8, CD28, CD40 L, CD44, CD47, CD137, OX40 or TGFβ.

14. A pharmaceutical composition, comprising:
the antibody or the antigen-binding portion thereof of claim 1; and
at least one pharmaceutically acceptable carrier.

15. The pharmaceutical composition of claim 14, further comprises an additional therapeutic agent.

16. A method of treating a cancer in a human, comprising: using the antibody, or an antigen-binding portion thereof of claim 1.

17. The method of claim 16, wherein the cancer comprises breast cancer, lung cancer, prostate cancer, pancreatic cancer, gastric cancer, ovarian cancer and endometrial cancer and colon cancer, liver cancer, nasopharyngeal cancer, skin cancer, oral cancer, renal cancer, brain cancer, cervical cancer and bladder cancer.

18. A method of treating cancer in a human, comprising:
administering to a subject in need thereof an effective amount of the pharmaceutical composition comprising the antibody or the antigen-binding portion thereof of claim 1, and/or a combination of a chemotherapeutic agent, a photodynamic therapeutic agent or a biological agent.

19. The method of claim 18, wherein the combination provides a synergistic effect in inducing or enhancing immune reaction.

20. A hybridoma designated as 1E12b deposited under ATCC Accession Number PTA-126149.

21. (canceled)

22. (canceled)


SEQUENCE LISTING (10 sequences)

SEQ ID NO: 1 - DNA, 360 bp, Artificial Sequence
  Heavy Chain Variable Region (VH)
gaggtgaagcttctcgagtctggaggtggcctggtgcagcctggaggatccctgaaactctcctgtgcagcctcaggattcgattttagtatatactggatgagttgggtccggcaggctccagggaaagggctagaatggattggagaaattaatcaacatagcagtacgataaactatacgccatctctaaaggataagttcatcatctccagagacaacgccaaaaatacgctgtacctgcaaatgagcaaagtgagatctgaggacacagccctttattactgtgcaagacggtatggtaactacgcagactactttgactactggggccaaggcaccactctcacagtctcctca

SEQ ID NO: 2 - DNA, 327 bp, Artificial Sequence
  Light Chain Variable Region (VL)
gaaaatgtgctcacccagtctccagcaatcatgtctgcatctccaggggaaaaggtcaccatgacctgcagggccagctcaagtgtaagttccaattacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccaccagtgcagtacttacccactcacgttcggtgctgggaccaagctggaactgaaacgg

SEQ ID NO: 3 - protein, 120 aa, Artificial Sequence
  Heavy Chain Variable Region (VH)
EVKLLESGGGLVQPGGSLKLSCAASGFDFSIYWMSWVRQAPGKGLEWIGEINQHSSTINYTPSLKDKFIISRDNAKNTLYLQMSKVRSEDTALYYCARRYGNYADYFDYWGQGTTLTVSS

SEQ ID NO: 4 - protein, 109 aa, Artificial Sequence
  Light Chain (VL)
ENVLTQSPAIMSASPGEKVTMTCRASSSVSSNYLHWYQQKSGASPKLWIYSTSNLASGVPARFSGSGSGTSYSLTISSVEAEDAATYYCHQCSTYPLTFGAGTKLELKR

SEQ ID NO: 5 - protein, 10 aa, Artificial Sequence
  Heavy Chain CDR1
GFDFSIYWMS

SEQ ID NO: 6 - protein, 14 aa, Artificial Sequence
  Heavy Chain  CDR2
EINQHSSTINYTPS

SEQ ID NO: 7 - protein, 11 aa, Artificial Sequence
  Heavy Chain CDR3
RYGNYADYFDY

SEQ ID NO: 8 - protein, 13 aa, Artificial Sequence
  Light Chain CDR1
RASSSVSSNYLHW

SEQ ID NO: 9 - protein, 7 aa, Artificial Sequence
  Light Chain CDR2
STSNLAS

SEQ ID NO: 10 - protein, 9 aa, Artificial Sequence
  Light Chain CDR3
CHQCSTYPL